(19) United States
(12) Patent Application Publication
(10) Pub. No.: US 2016/0090586 A1
(43) Pub. Date: Mar. 31, 2016

(54) ELECTRICAL STIMULATION APPARATUS

(71) Applicant: Samsung Electronics Co., Ltd., Suwon-si (KR)

(72) Inventors: Minseoks Kim, Yongin-si (KR); Younsuk Choi, Yongin-si (KR)

(21) Appl. No.: 14/871,585

(22) Filed: Sep. 30, 2015

(30) Foreign Application Priority Data
Sep. 30, 2014 (KR) 10-2014-0132016

(51) Int. Cl. C12N 13/00; G01N 33/483; G01N 27/00

(52) U.S. Cl. CPC C12N 13/00; G01N 27/00; G01N 33/4836

(57) ABSTRACT

An electrical stimulation apparatus including a plurality of stimulation units that provide electrical stimulations to a target material disposed in a chamber that receives the target material and a culture medium, wherein each of the plurality of stimulation units comprises a target region on which the target material is disposed, and a first electrode and a second electrode disposed spaced apart from each other, having the target region therebetween, and at least two of the plurality of stimulation units provides different electrical stimulations to the target material.

### RELATED APPLICATION

This application claims the benefit of Korean Patent Application No. 10-2014-0132016, filed on Sep. 30, 2014, in the Korean Intellectual Property Office, the disclosure of which is incorporated herein in its entirety by reference.

### BACKGROUND

1. Field

The present disclosure relates to an apparatus for providing electrical stimulation to a target material.

2. Description of the Related Art

Generally, analysis of the physical properties of cells is often conducted in disease diagnosis, medicinal efficacy testing, toxicity testing, and so forth. To analyze the characteristics of a cell, in the related art, an optical measurement method has been mainly executed, in which a cancer cell is treated with an anticancer drug and then the fluorescence of the cell is analyzed after exposure to the drug, in an in-vitro manner.

To improve the reliability of cell characteristic analysis, a method of measuring electrical characteristics of cells in addition to the optical measurement method is desired.

### SUMMARY

Provided is an electrical stimulation apparatus for providing a plurality of electric stimulations, wherein the electrical stimulation apparatus comprises a plurality of stimulation units that provide electrical stimulations to a target material, wherein the stimulation units are disposed in a chamber that receives the target material and a cell culture medium, wherein,

each of the plurality of stimulation units comprises a target region, on which the target material is disposed, and a first electrode and a second electrode spaced apart from each other, having the target region therebetween, and

at least two of the plurality of stimulation units provides different electrical stimulations to the target material.

Provided is an electrical stimulation apparatus for providing a plurality of electric stimulations, wherein the electrical stimulation apparatus comprises a first substrate on which a plurality of stimulation units that provide electrical stimulations to a target material are disposed; and

a second substrate forming a chamber that receives the target material by being coupled with the first substrate,

wherein each of the plurality of stimulation units comprises a target region on which the target material is disposed and a first electrode and a second electrode spaced apart from each other, having the target region therebetween, and

at least two of the plurality of stimulation units provides different electrical stimulations to the target material.

Additional aspects will be set forth in part in the description which follows and, in part, will be apparent from the description, or may be learned by practice of the presented exemplary embodiments.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 is an exploded perspective view schematically illustrating an electrical stimulation apparatus;

FIG. 2 illustrates the electrical stimulation apparatus illustrated in FIG. 1 when the elements shown in exploded-view are coupled;

FIG. 3 is a planar view illustrating a first substrate and a plurality of stimulation units;

FIG. 4 is a graph that illustrates the effect of the distance between a first electrode and a second electrode on uniformity of an electric field and average electric field strength plotted against;

FIG. 5 is a graph that illustrates the effect of the length of an electrode on degree of uniformity of an electric field and average electric field strength;

FIG. 6 is a graph illustrating the effect of the height of culture medium on degree of uniformity of an electric field and an average electric field strength;

FIG. 7 illustrates a plurality of stimulating units arranged two-dimensionally;

FIG. 8 is a planar view of a first substrate and a second substrate (illustrated in exploded view in FIG. 1) coupled;

FIG. 9 is a perspective view of part of a first substrate and a second substrate (illustrated in exploded view in FIG. 1) coupled;

FIG. 10 is a side view illustrating a second substrate without a flow path according to another exemplary embodiment;

FIG. 11 is a side view illustrating a first through third substrates (illustrated in exploded view in FIG. 1) that are coupled;

FIG. 12 is a planar view illustrating a fourth substrate having a heat-dissipation function (heat dissipation grooves) according to an exemplary embodiment; and

FIG. 13 illustrates a state where the fourth substrate illustrated in FIG. 12 and first and second substrates (illustrated in exploded view in FIG. 1) are coupled.

### DETAILED DESCRIPTION

According to an aspect of an exemplary embodiment, provided is an electrical stimulation apparatus including a plurality of stimulation units providing electrical stimulations to a target material and being disposed in a chamber that receives the target material and a culture medium. Each of the plurality of stimulation units comprises a target region on which the target material is disposed, and a first electrode and a second electrode spaced apart from each other with the target region positioned therebetween At least two of the plurality of stimulation units provides different electrical stimulations to the target material.

The electrical stimulations may be provided by a voltage applied between the first electrode and the second electrode.

The target region may be formed through a surface-treatment of a substrate with a material that facilitates adhesion of the target material.

The first electrode and the second electrode may be arranged symmetrically with respect to the target region.

The first electrode and the second electrode may be arranged on a same substrate as the target region, and longitudinal dimensions (e.g., the largest dimension) of the first electrode and the second electrode may be in parallel with one another and/or parallel to the surface of the substrate on which the target material is formed.

A distance between the first electrode and the second electrode may be longer than the maximum width of the target region, such as about 1.2 or more times as long as a maximum width of the target region, wherein the “width” of the target region is the dimension of the target region in a direction parallel to the direction of electrical stimulation between the first and second electrodes.

The plurality of stimulation units may include a first stimulation unit and a second stimulation unit that are arranged in parallel with one another with respect to the direction of electrical stimulation (the direction of electrical stimulation is the direction from the first electrode to the second electrode across the target region) and are adjacent to each other. Although the direction of electrical stimulation of adjacent stimulation units may be in parallel, the direction of an electrical stimulation provided by a first stimulation unit may be opposite to a direction of an electrical stimulation provided by an adjacent second stimulation unit.

Additionally, or alternatively, the plurality of stimulation units may include a first stimulation unit and a second stimulation unit (or third stimulation unit, etc.) that are arranged perpendicular to one another with respect to the directions of the electrical stimulation provided by the units, and are adjacent to each other. A direction of an electrical stimulation provided by the first stimulation unit may be the same as a direction of an electrical stimulation provided by the second stimulation unit (or third stimulation unit, etc.).

The electrical stimulation apparatus may further include a partition disposed between two or more (or between each) of the plurality of stimulation units. The partition may include a flow path through which the culture medium flows between the plurality of stimulation units. A height of the partition may be lower than a height of the chamber.

The electrical stimulation apparatus may further include a first electrode pad and a second electrode pad formed on a same plane (e.g., same surface) as (and connected to) the first electrode and the second electrode to apply a voltage received from outside (an external voltage source) to the first electrode and the second electrode, respectively. The first electrode pad and the second electrode pad are disposed outside the chamber.

The electrical stimulation apparatus may further include a circuit board generating a voltage to be applied to the first electrode and the second electrode and a first connection portion and a second connection portion disposed on the circuit board and electrically connected with a first electrode pad and a second electrode pad, respectively, through coupling between the circuit board and the chamber.

The electrical stimulation apparatus may further include a heat-dissipation member dissipating heat generated in the chamber outside. The heat-dissipation member may include, in a region corresponding to the first electrode and the second electrode, a channel through which a cooling fluid flows. The cooling fluid may be at least one of a gas and a liquid.

According to another aspect, an electrical stimulation apparatus is provided including a first substrate on which a plurality of stimulation units providing electrical stimulations to a target material are disposed, and a second substrate forming a chamber that receives the target material by being coupled with the first substrate, in which each of the plurality of stimulation units comprises a target region on which the target material is disposed, and a first electrode and a second electrode disposed apart from each other, having the target region therebetween, wherein at least two of the plurality of stimulation units provides different electrical stimulations to the target material.

The second substrate may include an opening in a region corresponding to the plurality of stimulation units.

The plurality of stimulation units may include a first stimulation unit and a second stimulation unit that are arranged in parallel with respect to the direction of the electrical stimulation and are adjacent to each other. A direction of an electrical stimulation provided by the first stimulation unit may be opposite to a direction of an electrical stimulation provided by the second stimulation unit.

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present inventive concept. Expressions such as “at least one of,” when preceding a list of elements, modify the entire list of elements and do not modify the individual elements of the list. The width and thickness of layers or regions illustrated in the appended drawings may be exaggerated for clarity. Throughout the detailed description, like reference numerals refer to like elements.

Electrical stimulations may be used for various purposes such as stem cell differentiation induction, circadian rhythm adjustment, reversible electroporation, irreversible electroporation, wound healing, induction of particular expression or protein secretion, Joule heating, and the like.

An electrical stimulation apparatus 10 according to an exemplary embodiment may provide electrical stimulations under various conditions to an adhesive cell that is being cultivated, and may be used as a screening apparatus for evaluating the effect of electrical stimulation applied to a cell, an apparatus for imaging the cell(the form and a quality of which change due to the applied electrical stimulation), and an electrical stimulation analyzing apparatus for separating a thermal effect.

In the following description, a target material is an object to which an electrical stimulation is applied, the physical characteristics of which may, in some instances, be changed by such electrical stimulations. The target material may be a biological material, for example, a cell, a micro cell, an exosome, a protein, a nucleic acid, a tissue, or the like, including combinations thereof.

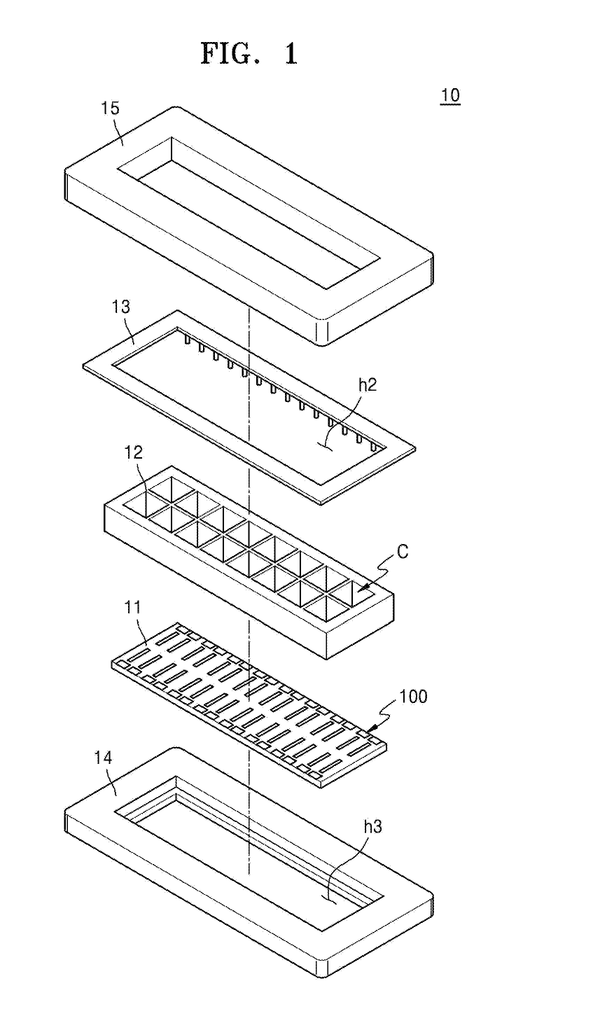

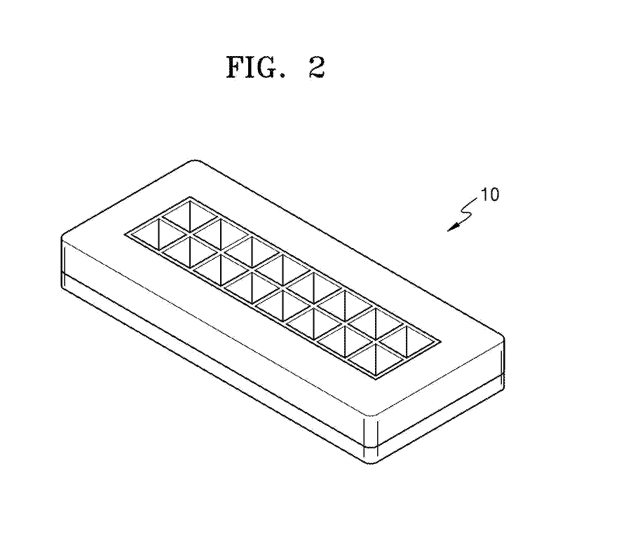

FIG. 1 is an exploded perspective view schematically illustrating an electrical stimulation apparatus according to an exemplary embodiment, and FIG. 2 illustrates the electrical stimulation apparatus illustrated in FIG. 1 in which the elements are coupled. As illustrated in FIGS. 1 and 2, the electrical stimulation apparatus 10 may include a first substrate 11 on which a plurality of stimulating units 100 are arranged; a second substrate 12 forming a chamber C together with the first substrate 11; a third substrate 13 electrically connected with the plurality of stimulating units 100; a fourth substrate 14 supporting the first substrate 11, and a cover 15 protecting, by covering, the electrical stimulation apparatus 10. Changes in the target material due to an electrical stimulation may be observed by a microscope under the electrical stimulation apparatus 10.

The first substrate 11 may be formed of a chemically and biologically inactive material. To observe the electric characteristics of the target material under the electrical stimulation apparatus 10, the first substrate 11 may be formed of a transparent material. For example, the first substrate 11 may be formed of various materials, such as acryl like polymethylmethacrylate (PMMA), polysiloxane like polydimethylsiloxane (PDMS), polycarbonate (PC), polyethylene like linear low-density polyethylene (LLDPE), low-density polyethylene (LDPE), medium-density polyethylene (MDPE), and high-density polyethylene (HDPE), polyvinyl alcohol, very low density polyethylene (VLDPE), polypropylene (PP), acrylonitrile butadiene styrene (ABS), a plastic material such as cyclo-olefin copolymer (COC), glass, mica, silica, semiconductor wafer, and the like, including combinations thereof. However, these materials are merely examples of a material for the first substrate 11, and useful materials are not limited thereto. Thus, any material may be used as the material of the first substrate 11 provided the material has chemical or biological stability, is chemically and biologically inactive, and mechanical processibility.

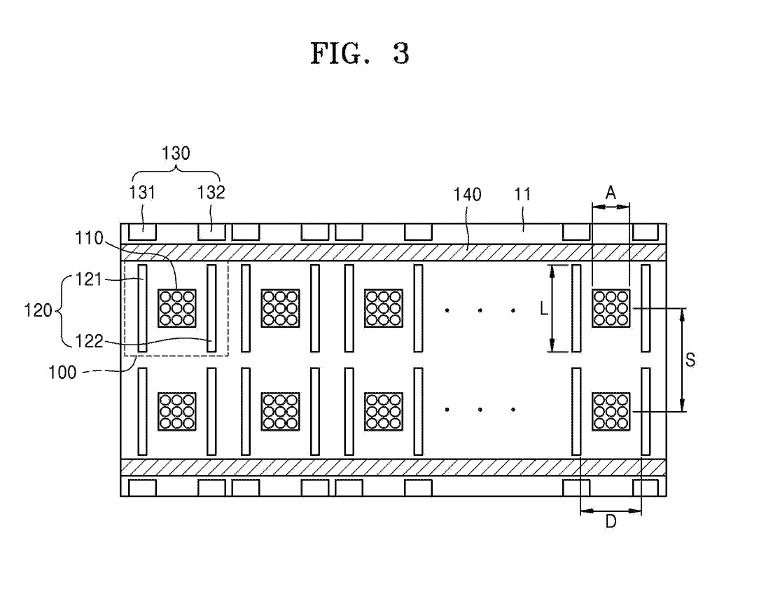

The plurality of stimulation units 100 may be arranged on the first substrate 11. FIG. 3 is a planar view illustrating the first substrate 11 and the plurality of stimulation units 100. The plurality of stimulation units 100 may be arranged one-dimensionally or two-dimensionally (e.g., in a one dimensional or two dimensional array, such as a single row or a plurality of rows). In FIG. 3, the plurality of stimulation units 100 arranged two-dimensionally are illustrated. Each stimulation unit 100 may include a target region 110 to which a target material adheres and an electrode pair 120 providing an electrical stimulation to the target region 110.

The target region 110 may be formed on the first substrate 11 through a surface-treatment with a material that facilitates adhesion of the target material. For example, a parylene coating may be applied onto the first substrate 11 to form the target region 110. The target region 110 may be formed on the first substrate 11 by using a photosensitive polymer. Thus, the target material introduced to the stimulation unit 100 or chamber leading thereto may be collected on the target region 110. However, the present disclosure is not limited thereto. The target region 110, for example, may be determined by first positioning the target material onto the substrate. For example, the target material may be dropped on the first substrate 11 through a micro nozzle capable of moving in parallel with the first substrate 11 of the electrical stimulation apparatus 10 on a stage on which the electrical stimulation apparatus 10 may be positioned. Then, a region onto which the target material is deposited may be the target region 110.

The shape of the target region 110 is not particularly limited. In FIG. 3, the target region 110 has a quadrilateral shape. The target region 110 may be a quadrangle having a width of about 2 mm. However, the present disclosure is not limited thereto. The target region 110 may be in a polygonal, circular, or oval shape as well as in a quadrilateral shape.

The electrode pair 120 may be patterned in the form of a thin film on the first substrate 11. The electrode pair 120 may include a first electrode 121 and a second electrode 122 that are disposed apart from each other, having the target region 110 positioned therebetween. The first electrode 121 and the second electrode 122 may be disposed symmetrically with respect to the target region 110. The distance “D” between the first electrode 121 and the second electrode 122 may be about 1.2 times or more as long as a maximum width A of the target region 110. Also, the distance D may be about 5 times or less as long as the maximum width A of the target region 110. For example, the distance D between the first electrode 121 and the second electrode 122 may be about 5 mm.

The first electrode 121 and the second electrode 122 may be in a polygonal shape and may be arranged in parallel with the first substrate 11. For example, the first electrode 121 and the second electrode 122 may have a rectangular shape having a narrow width and a long length, designated as “L” in FIG. 3. The first and second electrodes may have a length, for example, longer than the maximum width “A” of the target region 110, and may be about 5 times or less as long as the maximum width A of the target region 110. For example, the length L of the electrode may be about 5 mm. The width of the first electrode 121 and the second electrode 122 may be about 0.5 mm or less. The direction of an electric field formed between the first electrode 121 and the second electrode 122 may be parallel with the surface of the first substrate 11 upon which the electrodes and target region are disposed, and the electric field may be formed on the target region 110. As used herein, the direction of the electric field may refer to a direction of the average electric field.

Although the first electrode 121 and the second electrode 122 may have a rectangular shape in FIG. 3, they are not limited to this illustration and may have any shape if they may form a uniform electric field on the target region 110. As used herein, the uniform electric field may refer to an electric field having a strength that allows the target material to react to an electrical stimulation applied to the target material in the same manner, including, but not limited to, an electric field having a degree of uniformity of 100%. For example, even when the degree of uniformity of the electric field is about 85%, it may be said that a uniform electric field is formed if the target material reacts to the electrical stimulation in the same manner.

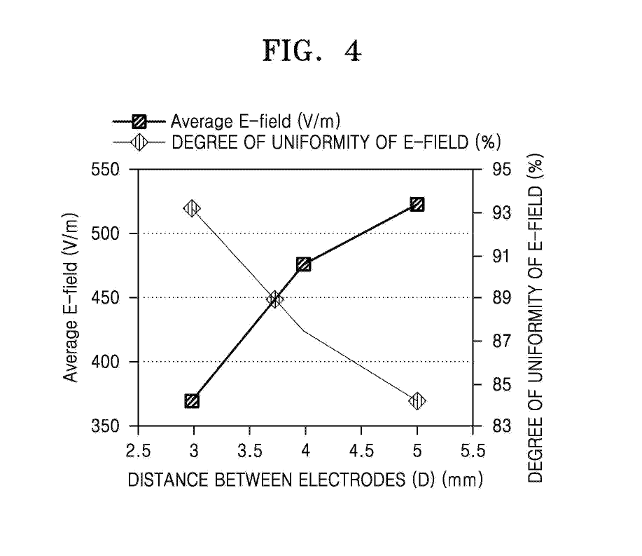

FIG. 4 illustrates the relationship between the degree of uniformity of an electric field and an average electric field with respect to a distance between a first electrode and a second electrode. An electrode used in stimulation has a rectangular shape with a narrow width and a long length (i.e., a width dimension that is smaller than the length dimension). As illustrated in FIG. 4, as a distance D between electrodes increases, the degree of uniformity of an electric field increases. However, as the distance D between the electrodes increases, an average electric field decreases, such that an electrical stimulation applied to a target material may be weakened. Thus, it is desirable to determine the distance D between the electrodes, such that the degree of uniformity of the electric field may be maintained in a predetermined range while maintaining a predetermined amount of average electric field applied to the target material.

To form a uniform electric field on the target region 110, the distance D between the first electrode 121 and the second electrode 122 may be about 1.2 times or more as long as a maximum width A of the target region 110. The distance D between the first electrode 121 and the second electrode 122 may be about 5 times or less as long as the maximum width A of the target region 110. For example, the distance D between the first electrode 121 and the second electrode 122 may be about 5 mm.

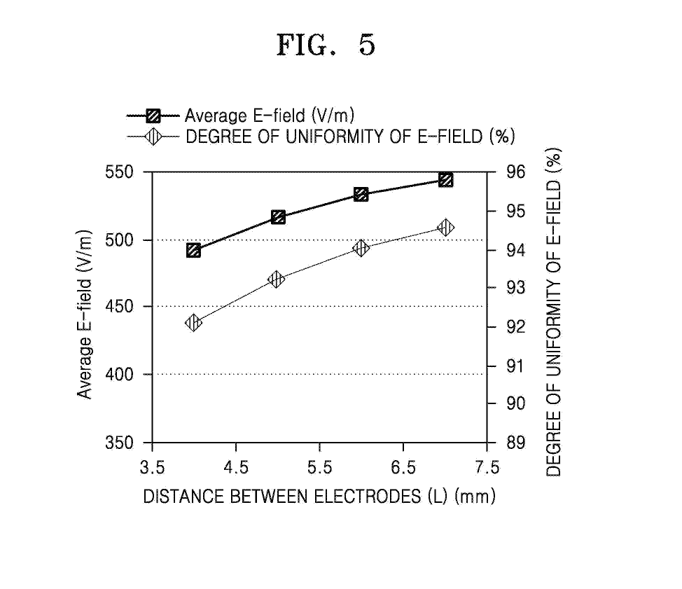

FIG. 5 illustrates simulation results of a relationship between the degree of uniformity of an electric field and an average electric field with respect to a length of an electrode. The electrode used in stimulation is in a rectangular shape having a narrow width and a long length. As illustrated in FIG. 5, as the length L of the electrode increases, the degree of uniformity of the electric field and the strength of the average electric field also increase. As such, as the length L of the electrode increases, a more uniform electric field may be formed.

Since the electrical stimulation apparatus 10 according to an exemplary embodiment needs to include the plurality of stimulation units 100, the length L of the electrode may be limited. The length L of the electrode according to an exemplary embodiment may be longer than the maximum width A of the target region 110, and may be about 5 times or less as long as the maximum width A of the target region 110. For example, the length L of the electrode may be about 5 mm.

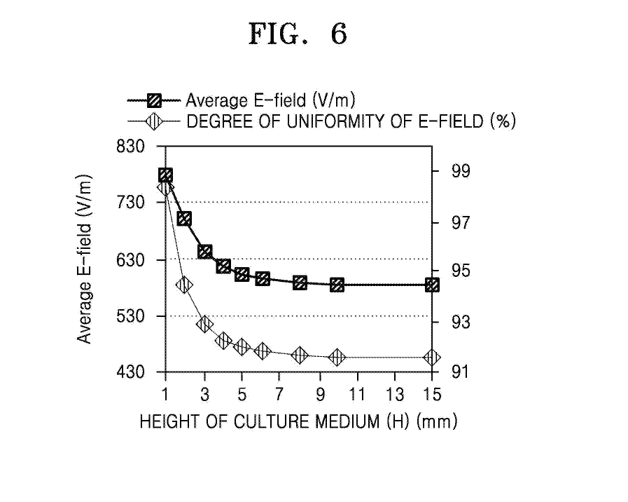

FIG. 6 illustrates simulation results of a relationship between the degree of uniformity of an electric field and an average electric field with respect to a height of a culture medium contained in the electrical stimulation apparatus 10. A Dulbecco's modified eagle medium (DMEM) having 5% fetal bovine serum (FBS) added thereto was used as a culture medium, and two electrodes, each of which has a width of 0.5 mm and a length L of 5 mm, are displaced spaced apart from each other by 5 mm. As illustrated in FIG. 6, as the height of the culture medium increases, the degree of uniformity of the electric field and the strength of the average electric field decrease. The degree of uniformity of the electric field and the strength of the average electric field decrease inversely proportionally to the height of the culture medium and converge at a predetermined value. Thus, by maintaining the culture medium of the predetermined height, the uniform electric field may be formed.

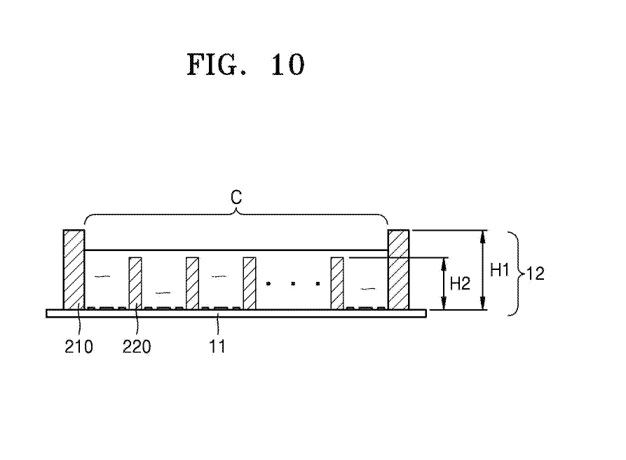

If the height of the culture medium is low, a variation in each of the degree of uniformity of the electric field and the strength of the average electric field may increase. Thus, the height of the culture medium according to an exemplary embodiment may be about 5 mm to about 15 mm as measured from the surface of the first substrate 11. In addition, to saturate the strength of the electric field, a height “H,” as shown in FIG. 10 of the culture medium may be about 1.5 times to about 2.5 times as high as the distance D between the electrodes.

Referring back to FIG. 3, a first electrode pad 131 and a second electrode pad 132, which provide an electrode pad pair 130, may be disposed on the first substrate 11 to receive an electric signal from the third substrate 13 (as shown in FIG. 1) and to deliver the electric signal to the first electrode 121 and the second electrode 122 of the stimulation unit 100. The first and second electrode pads 131 and 132 corresponding to the first and second electrodes 121 and 122 may be formed by one-time patterning with the same conductive material. The conductive material, except for the conductive material of the electrodes 121 and 122 and the electrode pads 131 and 132 corresponding thereto, is covered with an insulating material layer 140, such that the electrodes 121 and 122 and their corresponding electrode pads 131 and 132 may be distinguished from other features present on the substrate.

The electrodes 121 and 122 may be directly connected to the electrode pads 131 and 132. That is, the insulating material layer 140 may not cover the conductive material. However, by covering the conductive material with the insulating material layer 140, instead of exposing the conductive material as a whole, an electric field forming factor for the target region 110 may be limited to the electrodes 121 and 122, thus improving the degree of uniformity of the electric field on the target region 110.

The electrodes 121 and 122 and the electrode pads 131 and 132 according to an exemplary embodiment may be formed of a conductive material, and may be formed of a metallic or conductive metallic oxide. For example, the electrode may be formed of metal such as Ti, Pt, Ru, Au, Ag, Mo, Al, W, or Cu or a metallic oxide such as indium tin oxide (ITO), aluminum zinc oxide (AZO), indium zinc oxide (IZO), tin oxide (SnO₂) or In₂O₃. However, the aforementioned materials are merely examples of a material for the electrodes 121 and 122 and the electrode pads 131 and 132, and exemplary embodiments are not limited thereto.

The plurality of stimulation units 100 may independently apply electrical stimulation to the target material. For example, at least two of the plurality of stimulation units 100 may provide different electrical stimulations from each other. The electrical stimulation may be provided by a voltage applied between the first electrode 121 and the second electrode 122. The different electrical stimulations from each other may be electrical stimulations having different polarities from each other or electrical stimulations having different amounts (e.g., voltages) from each other. The voltage may be applied as a pulse type. An electric field may be formed on the target region 110 by the voltage between the first electrode 121 and the second electrode 122. A target material disposed on each stimulation unit 100 may independently react to the electrical stimulation. Thus, the plurality of stimulation units 100 may be arranged so as not to cause electrical interference therebetween. As described previously, the plurality of stimulation units 100 may be arranged in a single row or a plurality of rows. For example, the plurality of stimulation units 100 may be arranged in parallel with or perpendicular to the direction of an electric field formed thereon.

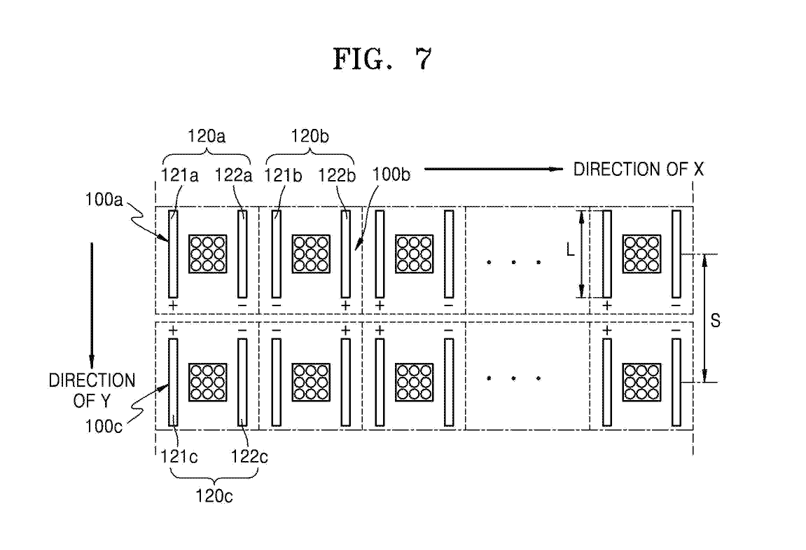

FIG. 7 illustrates a part of the stimulation units 100 arranged in two rows according to an exemplary embodiment. As illustrated in FIG. 7, to reduce electrical interference, the stimulation units 100 may be arranged in such a way that a distance S between the centers of the stimulation units 100 is about two times or more as long as the distance D (as illustrated in FIG. 3) between the electrodes in the same stimulation unit 100. A voltage may be applied to each stimulation unit 100 in such a way that the electric field directions on adjacent stimulation units 100 among the stimulation units 100 arranged in parallel with the electric field direction (in the direction of X) are opposite to each other. For example, a second electrode 122a of a first stimulation unit 100a and a first electrode 121b of a second stimulation unit 100b may be applied with a voltage having the same polarity. Since the adjacent stimulation units 100a and 100b are arranged such that the electric field directions of the adjacent stimulation units 100a and 100b become opposite to each other, electrical interference between the stimulation units 100a and 100b may be reduced.

Then, a voltage may be applied to each stimulation unit 100 in such a way that the electric field directions of the adjacent stimulation units 100 among the stimulation units 100 arranged perpendicularly to the electric field direction (in the direction of Y) are the same as each other. For example, a voltage having the same polarity may be applied to the first electrode 121a of the first stimulation unit 100 and a first electrode 121c of a third stimulation unit 100c, and a voltage having the same polarity may be applied to the second electrode 122a of the first stimulation unit 100a and a second electrode 122c of the third stimulation unit 100c. By arranging the adjacent first and third stimulation units 100a and 100c to have the same electric field direction, electrical interference between the first and third stimulation units 100a and 100c may be reduced.

As illustrated in FIG. 10, the second substrate 12 may form the chamber C by being coupled with the first substrate 11. The first substrate 11 and the second substrate 12 may be engaged with each other using a pressure scheme. For example, an O-ring may be interposed between the first substrate 11 and the second substrate 12, which are then pressurized to form the chamber C. The chamber C may have a height capable of receiving a culture medium and target material.

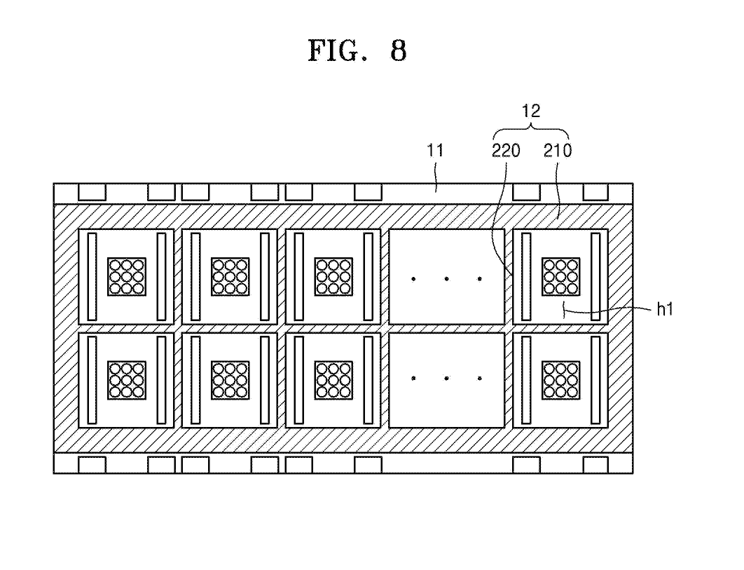

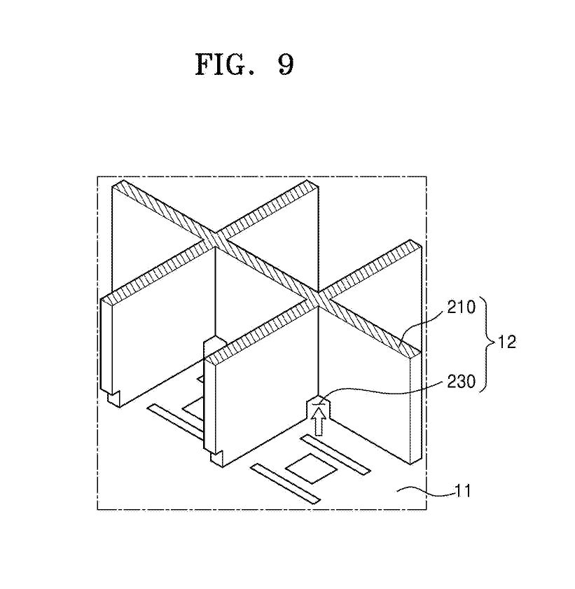

FIG. 8 is a planar view illustrating a state where the first substrate 11 and the second substrate 12 illustrated in FIG. 1 are coupled, and FIG. 9 is a part of a perspective view illustrating a state where the first substrate 11 and the second substrate 12 illustrated in FIG. 1 are coupled. As illustrated in FIG. 10, the second substrate 12 may have a mesh structure including a plurality of openings h1. The opening h1 may correspond to (i.e., overlap with) the stimulation unit 100, as is shown in FIG. 1. If the plurality of stimulation units 100 are arranged in a single row, the plurality of openings h1 may be arranged in a single row, and if the plurality of stimulation units 100 are arranged in a plurality of rows, the plurality of openings h1 may also be arranged in a plurality of rows. The size of the opening h1 may correspond to that of the stimulation unit 100. Although the opening h1 is in a quadrilateral shape, the shape of the opening h1 is not limited thereto. The shape of the opening h1 may have at least one of a circular shape, an oval shape, and a polygonal shape. The size of the opening h1 may be or may not be uniform.

The second substrate 12 may be divided by a first partition 210 forming the edge of the second substrate 12 and a second partition 220 disposed inside the second substrate 12. The chamber C may be formed by coupling the first substrate 11 and the first partition 210. When the first substrate 11 and the first partition 210 are coupled, the stimulation unit 100 may be positioned inside the first partition 210 and the electrode pad pair 130 may be positioned outside the first partition 210. A flow path such as the flow path illustrated in FIG. 9, for introducing or discharging the culture medium may be formed in a region of the first partition 210.

As is illustrated in FIG. 10, the second partition 220 is disposed inside the second substrate 12 to partition the stimulation units 100. The second partition 220 may prevent electrical interference between the stimulation units 100. In the second partition 220, a flow path 230 may be formed to allow the culture medium to flow between the stimulation units 100. Thus, the culture medium may be filled in every stimulation unit 100 through the flow path 230.

The flow path 230 along which the culture medium may be omitted according to circumstances. FIG. 10 illustrates the second substrate 12 having no flow path according to another exemplary embodiment. As illustrated in FIG. 10, when the second partition 220 has no flow path 230, the effect of blocking electrical interference between the stimulation units 100 may increase. A height H1 of the first partition 210 may be equal to or different from a height H2 of the second partition 220. For example, the height H1 of the partition 210 may be greater than the height H2 of the second partition 220. As the chamber C is formed by being coupled with the first substrate 11, the first partition 210 may have the height H1 that is greater than the height of the culture medium. The second partition 220 may have the height H2 that is less than the height of the culture medium to facilitate flow of the culture medium between the stimulation units 100. Thus, even when being introduced into a first stimulation unit 100, the culture medium may be filled in the other stimulation units 100.

The second substrate 12 may be formed of a chemically and biologically inactive material which is also an insulating material for blocking electrical interference between the stimulation units 100. For example, the second substrate 12 may be formed of acryl such as polymethylmethacrylate (PMMA), polysiloxane like polydimethylsiloxane (PDMS), polycarbonate (PC), polyethylene such as linear low-density polyethylene (LLDPE), low-density polyethylene (LDPE), medium-density polyethylene (MDPE), and high-density polyethylene (HDPE), polyvinyl alcohol, very low density polyethylene (VLDPE), polypropylene (PP), acrylonitrile butadiene styrene (ABS), a plastic material such as cyclo-olefin copolymer (COC), polyether ether ketone (PEEK), glass, mica, silica, or the like. However, these materials are merely examples of materials for the second substrate 12, and exemplary embodiments are not limited thereto. Any material may be used for the second substrate 12 according to an exemplary embodiment as long as it has chemical and biological stability and insulating characteristics.

The third substrate 12 may include one or more source (e.g., chips) for generating a voltage to apply an electrical stimulation, for example, the voltage, to the stimulation unit. The third substrate 13 may be, but not limited to, an on-demand application specific integrated circuit (ASIC), a printed circuit board (PCB), or the like. The third substrate 13 may also also be referred to as a circuit board for generating an electrical stimulation. For example, the third substrate 12 may include pulse-wave generators or other periodic wave generator generating different pulse wave voltages for supplying to the various stimulation units.

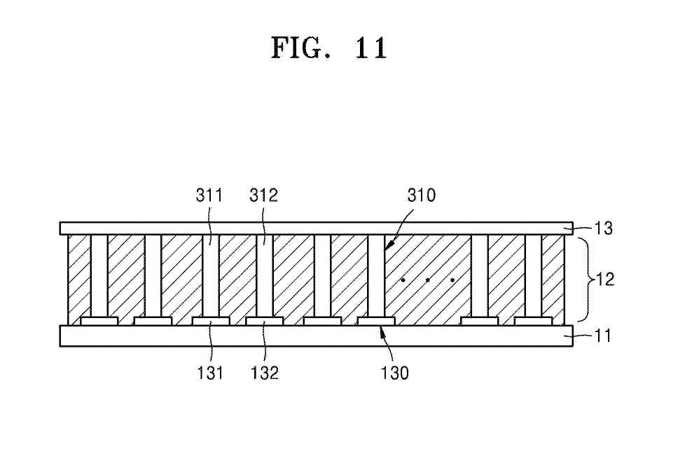

FIG. 11 is a side view illustrating a state where first through third substrates illustrated in FIG. 1 are coupled. As illustrated in FIG. 11, the third substrate 13 may include a plurality of connection portions 310 capable of being electrically connected with the stimulation unit 100. The third substrate 13 contacts the first substrate 11, having the second substrate 12 interposed therebetween, and thus the height of the plurality of connection portions 310 may be equal to or higher than the height of the first partition 210 of the second substrate 12. The plurality of connection portions 310 may protrude from the third substrate 13. The plurality of connection portions 310 may include a first connection portion 311 and a second connection portion 312 corresponding to the first electrode pad 131 and the second electrode pad 132 of the first substrate 11, respectively. Thus, when the third substrate 13 is coupled to the first substrate 11, the first connection portion 311 and the second connection portion 312 contact the first connection pad 131 and the second electrode pad 132 of the first substrate 11, respectively. The connection portions 310 may be formed of a conductive material.

Referring back to FIG. 1, an opening h2 may be formed in the middle of the third substrate 13. The opening h2 may have a size corresponding to the size of the chamber C embodiment, but is not limited thereto. The opening h2 may not be formed in the third substrate 13. When an electrical reaction is observed under the electrical stimulation apparatus 10 through a microscope, the third substrate 13 may or may not be transparent.

The fourth substrate 14, together with the first substrate 11, may support the electrical stimulation apparatus 10. In the fourth substrate 14, an opening h3 may be formed in a region corresponding to the target region 110 to facilitate observation of the target region 110 from outside. However, an exemplary embodiment is not limited thereto. The fourth substrate 14 may be formed of a transparent material, without having the opening h3.

When a voltage is continuously applied to the stimulation unit 100, heat may be generated in the electrode pair 120 due to Joule heating. The generated heat may be delivered to the target region 110. The heat may act as a false positive and undesired background when a change in the target material with respect to an electrical stimulation is analyzed, because a thermal stimulation is also applied to the target material as well as the electrical stimulation.

The fourth substrate 14 may also function to dissipate the heat generated in the electrode pair 120 outside. The heat generated in the electrode pair 120 may be emitted to the outside of the apparatus in various ways, such as by using an air cooling scheme, a water cooling scheme, the Peltier effect, and the like. When the fourth substrate 14 has a heat-dissipation function, it may also be referred to as a heat-dissipation member.

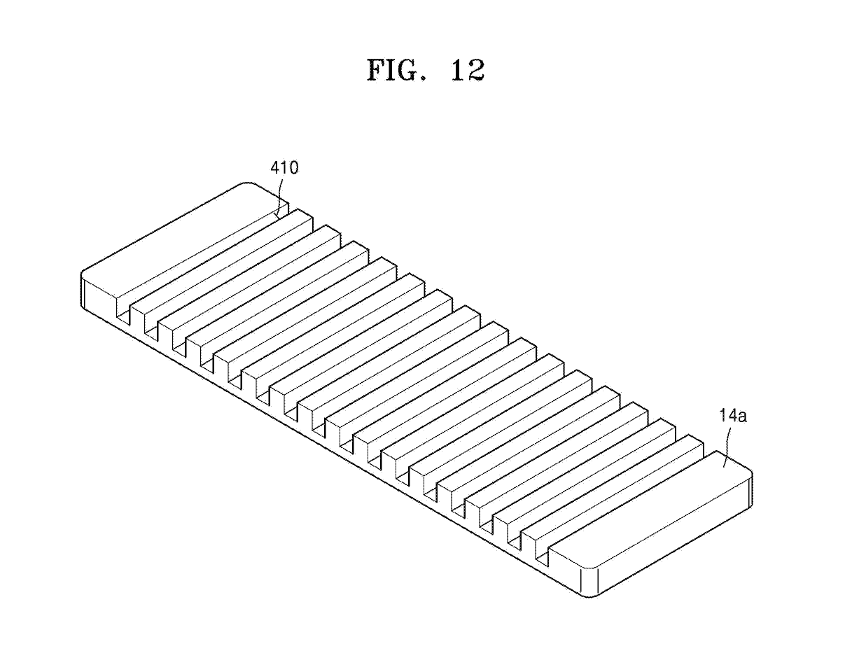

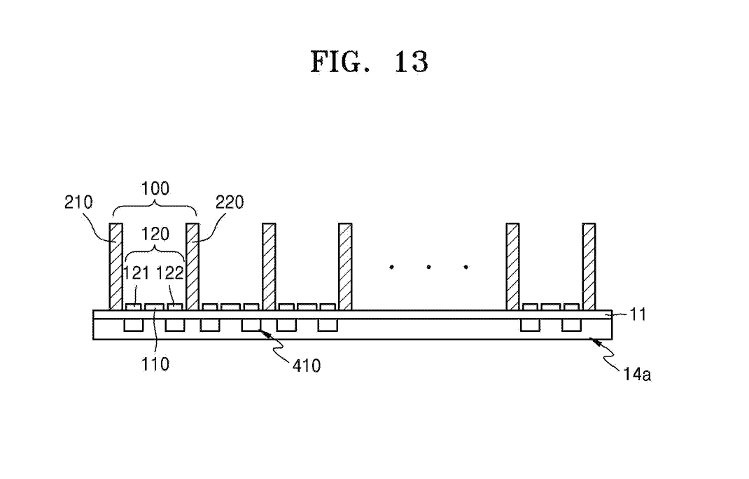

FIG. 12 is a planar view illustrating a fourth substrate 14a having a heat-dissipation function according to an exemplary embodiment, and FIG. 13 illustrates a state where the fourth substrate 14a illustrated in FIG. 12 and the first substrate 11 illustrated in FIG. 1 are coupled. As illustrated in FIGS. 12 and 13, the fourth substrate 14a may have formed therein a groove 410 through which a cooling fluid flows. The groove 410 may be positioned corresponding to the electrode pair 120. The groove 410 may become a channel due to coupling between the first substrate 11 and the fourth substrate 14. By allowing the cooling fluid to pass through the groove 410, that is, the channel, the heat of the electrical stimulation apparatus 10 may be dissipated and the temperature of the electrical stimulation apparatus 10 may be prevented from increasing. The cooling fluid may be a cooling gas or a cooling liquid.

Although it has been described that the groove may be formed in the fourth substrate to perform the heat-dissipation function, exemplary embodiments are not limited thereto. Thus, a channel along which the cooling fluid may flow may be formed in the fourth substrate.

The electrical stimulation apparatus according to an exemplary embodiment has the plurality of stimulation units in one chamber and independently provides different electrical stimulations to the plurality of stimulation units, thereby allowing a change of the target material with the electrical stimulations to be observed at high speed. Moreover, by removing a thermal stimulation, a change of an electrical stimulation may be accurately observed.

It should be understood that the exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

It should be understood that the exemplary embodiments described herein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An electrical stimulation apparatus comprising:
a plurality of stimulation units disposed in a chamber configured to receive a target material,
wherein each of the plurality of stimulation units comprises a target region that binds a target material, and a first electrode and a second electrode disposed apart from each other and having the target region positioned therebetween, and
wherein at least two of the plurality of stimulation units are configured to provide different electrical stimulations to the target material.

2. The electrical stimulation apparatus of claim 1, wherein the electrical stimulations are provided by a voltage between the first electrode and the second electrode.

3. The electrical stimulation apparatus of claim 1, wherein the target region comprises a material that adheres to a cell, an exosome, a protein, or a nucleic acid.

4. The electrical stimulation apparatus of claim 1, wherein the first electrode and the second electrode are arranged symmetrically with respect to the target region.

5. The electrical stimulation apparatus of claim 1, wherein the first electrode and the second electrode are arranged on a same substrate as the target region, and longitudinal dimensions of the first electrode and the second electrode are in parallel with the surface of the substrate on which the electrodes are arranged.

6. The electrical stimulation apparatus of claim 1, wherein the distance between the first electrode and the second electrode is about 1.2 or more times the maximum width of the target region.

7. The electrical stimulation apparatus of claim 1, wherein lengths of the first electrode and the second electrode are longer than the maximum width of the target region.

8. The electrical stimulation apparatus of claim 1, wherein the plurality of stimulation units comprise a first stimulation unit and a second stimulation unit that are arranged in parallel to each other with respect to the direction of electrical stimulation and are adjacent to each other, and
the direction of an electrical stimulation provided by the first stimulation unit is opposite to the direction of an electrical stimulation provided by the second stimulation unit.

9. The electrical stimulation apparatus of claim 1, wherein the plurality of stimulation units comprise a first stimulation unit and a second stimulation unit that are arranged perpendicular to one another with respect to the direction of electrical stimulation and are adjacent to each other, and
the direction of an electrical stimulation provided by the first stimulation unit is the same as the direction of an electrical stimulation provided by the second stimulation unit.

10. The electrical stimulation apparatus of claim 1, further comprising a partition disposed between each of the stimulation units.

11. The electrical stimulation apparatus of claim 10, wherein the partition comprises a flow path through which the culture medium flows between the plurality of stimulation units.

12. The electrical stimulation apparatus of claim 10, wherein a height of the partition is lower than a height of the chamber.

13. The electrical stimulation apparatus of claim 1, further comprising a first electrode pad and a second electrode pad that are formed on a same plane as the first electrode and the second electrode to apply a voltage received from an outside voltage source to the first electrode and the second electrode, respectively,
wherein the first electrode pad and the second electrode pad are disposed outside the chamber.

14. The electrical stimulation apparatus of claim 1, further comprising:
a circuit board that generates a voltage to be applied to the first electrode and the second electrode; and
a first connection portion and a second connection portion disposed on the circuit board and electrically connected with a first electrode pad and a second electrode pad, respectively, through coupling between the circuit board and the chamber.

15. The electrical stimulation apparatus of claim 1, further comprising a heat-dissipation member that dissipates heat generated in the chamber.

16. The electrical stimulation apparatus of claim 15, wherein the heat-dissipation member comprises a channel through which a cooling fluid flows.

17. The electrical stimulation apparatus of claim 16, wherein the cooling fluid is a gas, a liquid, or a combination thereof.

18. The electrical stimulation apparatus of claim 1 further comprising:
a first substrate on which the plurality of stimulation units are disposed; and
a second substrate coupled with the first substrate to form the chamber configured to receive a target material.

19. The electrical stimulation apparatus of claim 18, wherein the second substrate comprises an opening in a region that corresponds to the plurality of stimulation units, such that the plurality of stimulation units are visible through the opening region of the second substrate.

20. The electrical stimulation apparatus of claim 18, wherein the plurality of stimulation units comprise a first stimulation unit and a second stimulation unit that are arranged in parallel to one another with respect to the direction of the electrical stimulations and are adjacent to each other, and
the direction of an electrical stimulation provided by the first stimulation unit is opposite to the direction of an electrical stimulation provided by the second stimulation unit.

21. A method of electrically stimulating a target material, the method comprising introducing a target material into the chamber of an electrical stimulation apparatus of claim 1, and applying a voltage across the first and second electrodes of the electrical stimulation apparatus to electrically stimulate the target material, wherein the target material is, optionally, a a cell, an exosome, a protein, or a nucleic acid.
